(19) United States
(12) Patent Application Publication
(10) Pub. No.: US 2006/0293740 A1
(43) Pub. Date: Dec. 28, 2006

(54) Epicardial lead fixation system

(75) Inventors: Ronald W. Heil JR., Roseville, MN (US); Geoffrey Willis, Redwood City, CA (US)

Correspondence Address: GUIDANT PATENT DOCKETING;FAEGRE & BENSON, LLP, 2200 WELLS FARGO CENTER, 90 SOUTH SEVENTH STREET, MINNEAPOLIS, MN, 55402-3901, US

(73) Assignee: Cardiac Pacemakers, Inc., St. Paul, MN (US)

(21) Appl. No.: 11/168,048

(22) Filed: Jun. 28, 2005

(51) Int. Cl. A61N 1/00

(52) U.S. Cl. 607/141

(57) ABSTRACT

A cardiac pacing lead for electrically communicating with a human heart is attached to the heart using an adhesive. The lead includes a flexible, elongated lead body having a proximal end and a distal end, a pad having an interface surface coupled to the lead body at the distal end. An electrode is coupled to the pad. The lead further includes an adhesive for bonding the pad to the heart such that the electrode is electrically coupled to the heart. The adhesive is delivered to the interface surface after the lead has been inserted into a subject. In one embodiment, the lead system includes an anchor having an interface surface adapted for adhesive coupling to the heart. In this embodiment, the anchor is coupled to a tether and the lead includes a lumen adapted to accept the tether and to allow advancement of the lead over the tether.

### TECHNICAL FIELD

The present invention relates to implantable lead assemblies for stimulating and/or sensing electrical signals in tissue. The present invention more particularly relates to myocardially and epicardially-implanted leads and systems for attaching the leads.

### BACKGROUND

Cardiac resynchronization therapy (“CRT”) (also commonly referred to as biventricular pacing) is an emerging treatment for congestive heart failure, which requires stimulation of both the right and the left ventricle to increase cardiac output. Left ventricular stimulation requires placement of a lead in or on the left ventricle near the apex of the heart. One technique for left ventricular lead placement is to expose the heart by way of a thoracotomy or a minimally-invasive technique. The lead is then positioned so that one or more electrodes contact the epicardium or are embedded in the myocardium. Another method is to advance an epicardial lead intravenously into the coronary sinus and then advance the lead through a lateral vein of the left ventricle.

The lead typically consists of a flexible conductor surrounded by an insulating tube or sheath that extends from the electrode at the distal end to a connector pin at the proximal end. The lead electrode at the distal end is attached to the heart tissue, such that the electrode is disposed adjacent the epicardial surface of the left ventricle. The left ventricle beats forcefully as it pumps oxygenated blood throughout the body. Repetitive beating of the heart can sometimes dislodge the lead from the myocardium or epicardium. The electrodes may lose contact with the heart muscle, or spacing between electrodes may alter over time. There is thus a need for a lead and attachment system that allows a lead to be securely attached to the myocardium or epicardium. There is a further need for an attachment system that is amenable to implantation in a minimally-invasive manner (e.g., minithoracotomy or subxiphoid approach).

### SUMMARY

The present invention, according to one embodiment is a lead for electrically communicating with a human heart. The lead comprises a flexible, elongated lead body having a proximal end and a distal end. A pad having an interface surface is coupled to the lead body at the distal end. An electrode is coupled to the pad. The lead further includes a delivery means for delivering an adhesive to the interface surface after the lead has been inserted into a subject, wherein the adhesive bonds the pad to the heart, such that the electrode is electrically coupled to the heart.

According to another embodiment, the present invention is a method of electrically coupling a cardiac rhythm management system, including a pulse generator and a cardiac lead, to a human heart. The method comprises providing a cardiac lead having a flexible, elongated lead body having a proximal end, a distal end, and an electrode coupled to the lead body near the distal end, the lead having an interface surface near the distal end adapted for interfacing with the heart, inserting the lead into the subject, and delivering an adhesive to the interface surface near the electrode, after inserting the lead and bonding the interface surface to the heart, such that the electrode is electrically coupled to an epicardial surface of the heart.

The present invention, according to another embodiment, is a lead for electrically communicating with a human heart. The lead comprises a flexible, elongated lead body having a proximal end and a distal end. The lead body has an exit slot located near the distal end and a lumen extending from the proximal end to the exit slot. An electrode is coupled to the distal end of the lead body. A tether having a tether proximal end and a tether distal end extends through the lumen. An anchor having an interface surface is adapted for coupling to the heart using an adhesive. The anchor is coupled to the tether distal end.

According to yet another embodiment, the present invention is a method of electrically coupling a cardiac pacing lead to a human heart. The method comprises providing a cardiac lead having a flexible, elongated lead body having a proximal end and a distal end, a lumen extending longitudinally through the lead body, and an electrode coupled near the distal end of the lead body, positioning an anchor on an epicardium of the heart, the anchor coupled to a longitudinally extending tether and having an interface surface adapted for coupling to the heart using an adhesive, delivering the adhesive to the interface surface to bond the anchor to the epicardium, advancing the cardiac lead over the tether toward a location on the heart near the anchor, and attaching the electrode to the heart.

While multiple embodiments are disclosed, still other embodiments of the present invention will become apparent to those skilled in the art from the following detailed description, which shows and describes illustrative embodiments of the invention. As will be realized, the invention is capable of modifications in various obvious aspects, all without departing from the spirit and scope of the present invention. Accordingly, the drawings and detailed description are to be regarded as illustrative in nature and not restrictive.

### BRIEF DESCRIPTION OF THE DRAWINGS

FIG. 1 shows a perspective view of an implantable cardiac function management system according to the present invention.

FIG. 2 shows a side plan view of an epicardial lead according to one embodiment of the present invention.

FIG. 3 shows a side plan view of an epicardial lead according to another embodiment of the present invention.

FIGS. 4A-4D show various views of an epicardial lead according to another embodiment of the present invention.

FIG. 5 shows a perspective view of an epicardial lead according to another embodiment of the present invention.

FIGS. 6A-6B show perspective views of an epicardial lead according to yet another embodiment of the present invention.

FIG. 7 shows a perspective view of a trans-myocardial attachment system according to another embodiment of the present invention.

FIG. 8 shows a cross-sectional view of a trans-myocardial attachment system according to another embodiment of the present invention.

FIG. 9 shows a cross-sectional view of a trans-myocardial attachment system according to another embodiment of the present invention.

FIGS. 10A-10F show various views of an epicardial attachment system according to another embodiment of the present invention.

FIG. 11 shows a method of using the epicardial attachment system of FIGS. 10A-10F.

While the invention is amenable to various modifications and alternative forms, specific embodiments have been shown by way of example in the drawings and are described in detail below. The intention, however, is not to limit the invention to the particular embodiments described. On the contrary, the invention is intended to cover all modifications, equivalents, and alternatives falling within the scope of the invention as defined by the appended claims.

### DETAILED DESCRIPTION

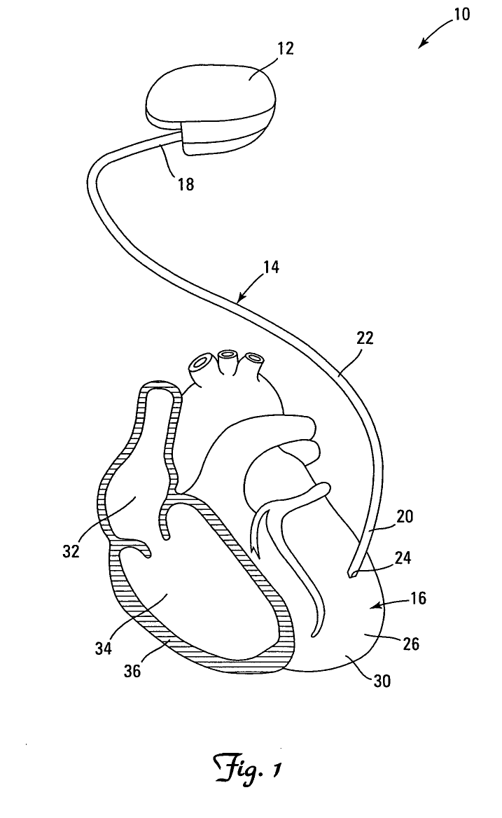

FIG. 1 is a perspective view of a cardiac function management system 10. As shown in FIG. 1, the system 10 includes a pulse generator 12 and a cardiac lead 14. The lead 14 operates to convey electrical signals between the heart 16 and the pulse generator 12. As shown, a proximal end 18 of the lead 14 is coupled to the pulse generator 12 and a distal end 20 is coupled to the heart 16. The lead 14 includes a lead body 22 extending from the lead proximal end 18 to the lead distal end 20.

As shown in FIG. 1, an electrode 24 is located at or near the distal end 20 of the lead 14 and is attached to the epicardium 26 of the heart 16. When positioned as above, the electrode 24 can be used to sense the electrical activity of the heart 16 or to apply a stimulating pulse to the left ventricle 30. In other embodiments, the cardiac lead 14 of the present invention can also be implanted in any other portion of the heart 16 as known in the art of cardiac rhythm or cardiac function management. For example, it may be implanted in the right atrium 32 or the right ventricle 34. The electrode 24 may alternatively be implanted in the myocardium 36 of the heart 16.

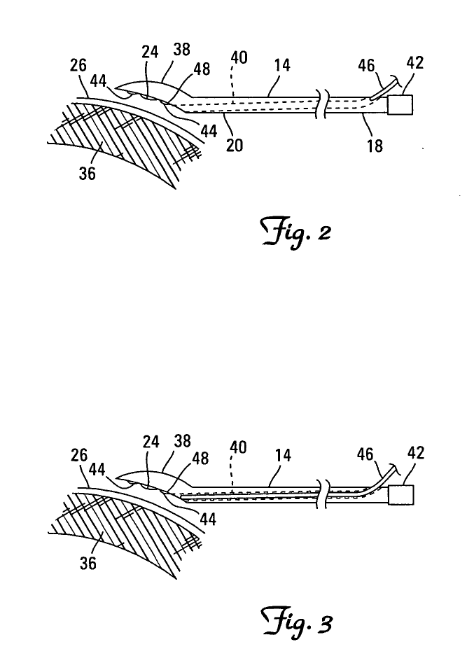

FIG. 2 shows a side plan view of an epicardial lead according to one embodiment of the present invention. In this embodiment, the lead 14 includes a pad 38 and a lumen 40 extending from the proximal end 18 to the distal end 20. A connector assembly 42, adapted to allow operative coupling of the electrode 24 and the pulse generator 12, is coupled to the lead body 22 at the proximal end 18. The lumen 40 is adapted to receive and carry an adhesive 44 through the lead 14 and deliver it to the pad 38. In one embodiment, the lead 14 further includes a removable adhesive catheter 46, which extends through the lumen 40. The adhesive catheter 46 is adapted to accept and convey the adhesive to the pad 38. The pad 38 may be formed from a silicone or any other suitable polymer. The adhesive 44 is distributed to an interface surface 48 of the pad 38 where it creates a bond between the epicardium 26 and the interface surface 48, thereby allowing for electrical contact between the electrode 24 and the epicardium 26. Some portions of the adhesive 44 may remain in the lumen 40, or the adhesive catheter 46, after delivery of the adhesive 44 to the pad 38.

The adhesive 44 may be any adhesive suitable for bonding with the heart tissue. Exemplary adhesives include BioGlue® (available from CryoLife, Inc. of Kennesaw, Ga.), fibrin sealants, cyanoacrylates, bovine collagen/thrombin, and polyethylene glycol polymers. Degradable or absorbable bio-compatible adhesives may be used, thereby leaving no permanent residual. Alternately, any permanent residual of a bio-compatible adhesive may be naturally encapsulated and rendered inert. Such adhesives may be cured or hardened via exposure to the environment, under the application of light, radiofrequency (RF) energy, or through the addition of a curing agent. One exemplary adhesive is one that cures at body temperatures. Another exemplary adhesive is a thermoset adhesive. Another appropriate light curing adhesive is the acrylic adhesive Loctite® 3321 (available from Loctite® of Rocky Hill, Conn.). Another category of adhesives are composed of natural proteins such as collagen or albumin. An adhesive is formed from these proteins by the addition of an aldehyde cross-linking additive, or other cross-linking additives as are known in the art.

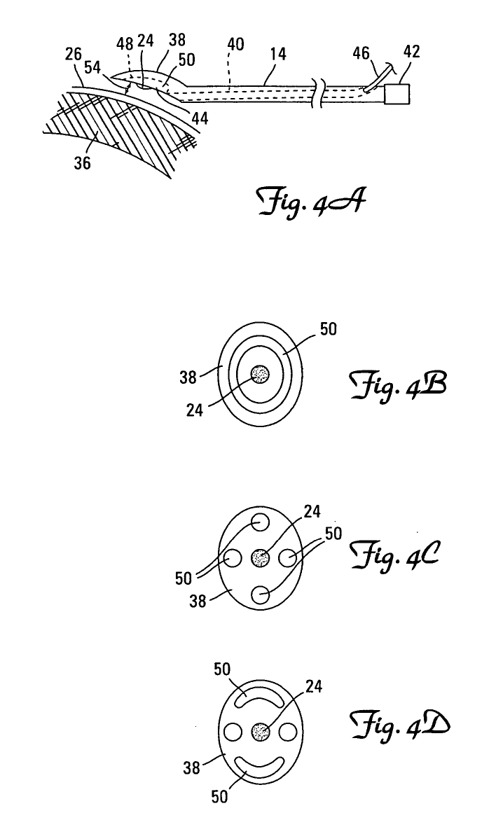

FIG. 3 shows a side plan view of another embodiment according to the present invention. In this embodiment, the catheter 46 is inserted through the lumen 40 from the proximal end 18 to the distal end 20. In this manner, the adhesive 44 is delivered to the pad 38 and the interface surface 48. After delivery, the catheter 46 can be removed from the lumen 40. This embodiment protects the lumen 40 from potential blockage by residual amounts of adhesive 44.

FIG. 4A shows a side plan view of an alternative embodiment according to the present invention. FIGS. 4B-4D show bottom plan views of the pad 38 of FIG. 4A. In the embodiments shown in FIGS. 4A-4B, the pad 38 includes a recess 50 adapted to retain the adhesive 44 and help to deliver it to the interface surface 48. In this embodiment, the adhesive 44 fills the recess 50, creating a bond between the interface surface 48 of the recess 50 and the epicardium 26. In this manner, a distance 54 between the pad 38 and the epicardium 26 can be minimized. In the embodiments shown in FIGS. 4A-4B, the cavity 50 has a substantially annular shape. Alternatively, the pad 38 could include multiple cavities 50 of a variety of shapes, including circular (see FIG. 4C) or semi-circular (see FIG. 4D). The pad 38 can include any number of cavities 50 of any shape suitable for bonding the pad 38 to the epicardium 26. The pad 38 shown in FIGS. 4A-4D has a substantially oval shape, but could have any shape that allows it to conform to the surface of the heart, including substantially circular, substantially square, or substantially rectangular. The adhesive 44 can be delivered through the lumen 40 as shown in FIG. 2 or through the catheter 46 inserted into the lumen 40 as shown in FIG. 3.

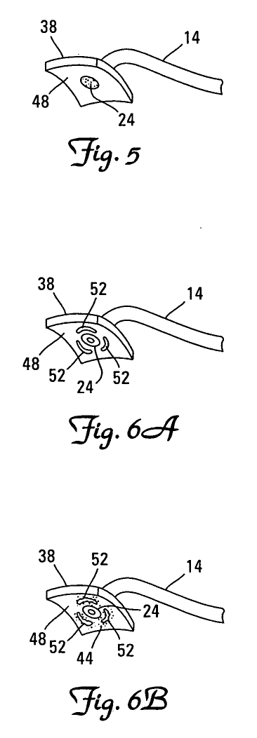

FIG. 5 shows a perspective view of an epicardial lead according to another embodiment of the present invention. In this embodiment, the pad 38 includes an electrode 24 and an interface surface 48. The electrode 24 is comprised of a porous material adapted for both absorbing the adhesive 44 and conveying electrical signals to the epicardium 26. Materials suitable for use as the porous material include generally biocompatible electrode materials such as platinum, titanium, platinum/iridium alloys, and other noble metal alloys. Alternatively, these materials could be coated with high surface area electrode treatments such as platinum “black,” titanium nitride, and iridium oxide. The porous material can allow the adhesive 44 to flow from the exterior to the interior of the electrode 24, or from the interior to the exterior of the electrode 24.

The lead 14 is attached to the electrode 24 and conveys electrical signals between the heart 16 and the electrode 24. In one embodiment, the electrode 24 is impregnated with the adhesive 44 using a separate catheter (not shown) to transport or deliver the adhesive to the electrode 24 or the interface surface 48 after the lead 14 has been advanced to the epicardium 26. In another embodiment, the adhesive is delivered through a lumen in the lead body 22 or a lumen and removable adhesive catheter as described with respect to FIG. 2. A bond is thereby created between the interface surface 48 and the epicardium 26. In one embodiment, the adhesive 44 comprises an electrically conductive material.

FIGS. 6A-6B are perspective views of another embodiment according to the present invention. In this embodiment, the pad 38 includes the electrode 24, the interface surface 48, and encapsulation pockets 52 containing adhesive 44. The electrode 24 may be comprised of a porous material. After the electrode 24 has been positioned in the desired location adjacent to the epicardium 26, the pad 38 is compressed against the epicardium 26 so that the encapsulation pockets 52 rupture, thereby distributing or delivering the adhesive 44 to the electrode 24 and the interface surface 48 and bonding them with the epicardium 26. Three encapsulation pockets 52 are shown in FIGS. 6A-6B, but any number, size, and shape of encapsulation pockets 52 could be used. The encapsulation pockets 52 can be comprised of a material that remains intact when dry, but upon exposure to the moist implant condition, degrades or dissolves, thus releasing the adhesive 44. Exemplary materials suitable for use in the encapsulation pockets 52 include gelatin, collagen, starch, or polysaccharides.

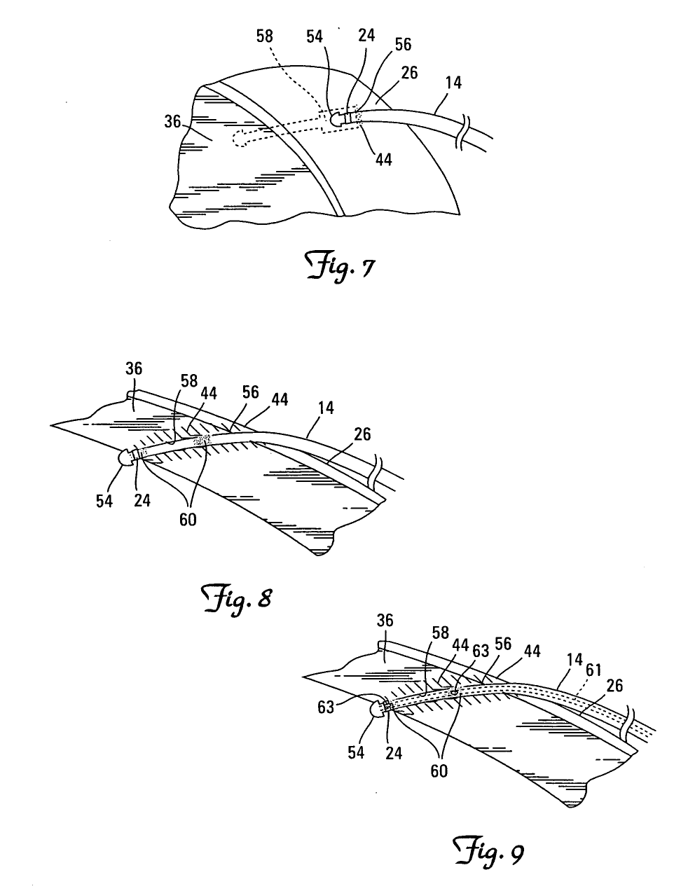

FIG. 7 is a perspective view of a trans-myocardial lead 14 according the present invention. In this embodiment, the electrode 24 is adapted for implantation in or through the myocardium 36. The electrode 24 may include a tip 54 adapted to puncture the epicardium 26 at puncture site 56 and form a tunnel 58 in the myocardium 36. In the embodiment shown in FIG. 7, the adhesive 44 is delivered to the puncture site 56 using an adhesive catheter (not shown) after the lead 14 is inserted into the subject. The adhesive catheter can be independently guided to the puncture site 56 or may be slideably guided using an endoscopic working channel to the puncture site 56 over the implanted lead body 22.

FIG. 8 is a cross-sectional view of an alternative embodiment of a trans-myocardial lead 14 according to the present invention. In this embodiment, the electrode 24 is implanted through the myocardium 36. The adhesive 44 is located on regions 60. The regions 60 act as interface surfaces for bonding the lead 14 and the electrode 24 to the myocardium 36. The adhesive 44 also aids in healing the wound created by insertion of the electrode 24. Optionally, the adhesive 44 can be injected into the tunnel 58 using an adhesive catheter.

FIG. 9 is a cross-sectional view of a yet another embodiment of a trans-myocardial lead according to the present invention. The lead 14 includes a lumen 61 and openings 63. The lumen 61 and the openings 63 allow the adhesive 44 to be delivered to the regions 60. The regions 60 then act as interface surfaces bonding the lead 14 and the electrode 24 to the myocardium 36. In one embodiment, the adhesive 44 can be delivered through the lumen 61 using an adhesive catheter (not shown). Alternatively, the adhesive 44 could be delivered to regions 60 before the lead is inserted into the subject. In this embodiment, the adhesive 44 can comprise a fibrin sealant that allows the electrode 24 and lead 14 to be inserted before the adhesive 44 sets up.

In an alternative trans-myocardial lead embodiment, a stylet (not shown) may be advanced through a lumen in the lead 14, such that it extends distal to a distal tip of the lead 14. The stylet may be adapted to puncture the epicardium 26 at puncture site 56 and form a tunnel 58 in the myocardium 36. Prior to advancing the lead 14, the adhesive 44 is injected into the tunnel 58. The adhesive 44 can also be delivered to the epicardial puncture site 56.

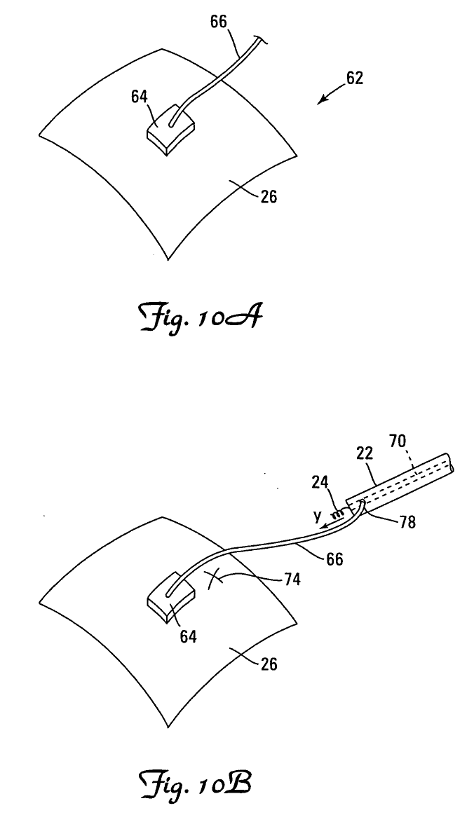

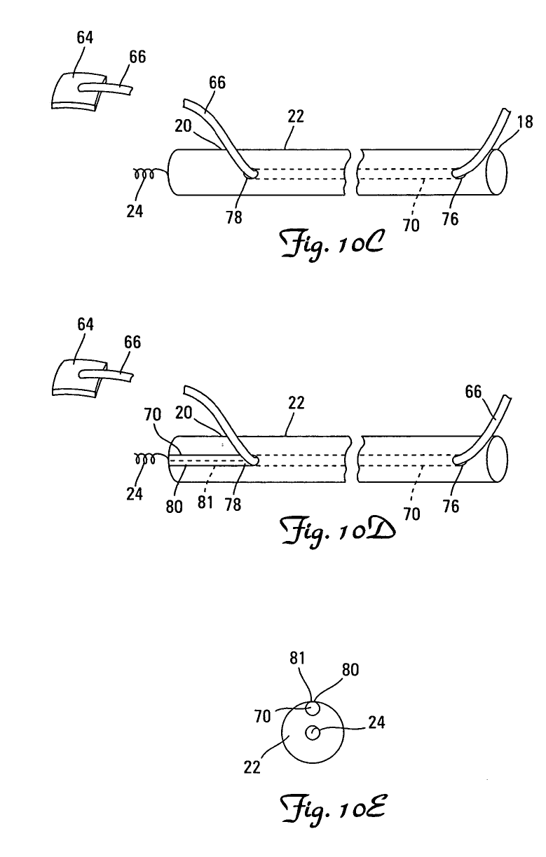

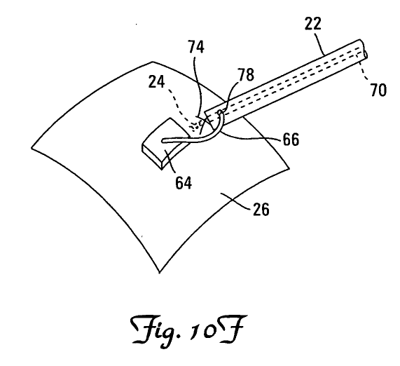

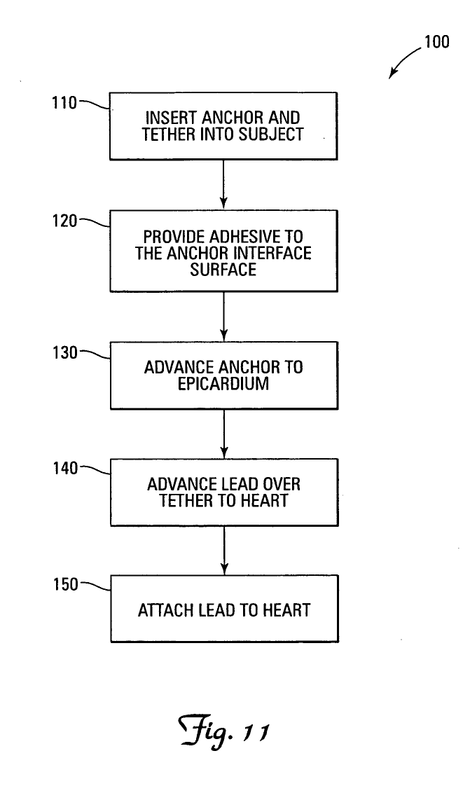

FIGS. 10A-10B show perspective views of an embodiment of an electrode attachment system 62. The attachment system 62 includes an anchor 64 and a tether 66. The anchor 64 is attached to the epicardium 26 by means of the adhesive 44. In one embodiment, the adhesive 44 is delivered via a separate adhesive catheter (not shown) after the anchor has been inserted into the subject. In another embodiment, the adhesive is contained in encapsulation pockets on the anchor 64, such as is described above with respect to FIGS. 6A-6B. As shown in FIG. 10B, the attachment system 62 further includes a lead body 22. The lead body 22 includes a lumen 70 for accepting the tether 66. The electrode 24 comprises an extendable/retractable helix that acts as a fixation means to attach the distal end of the lead 22 to the epicardium 26. In one embodiment, the anchor 64, the tether 66, or both are resorbable, such that over time they dissolve and are absorbed by the body. In this embodiment, fixation of the lead 14 is ultimately accomplished first by the helical electrode 24 at the time of implantation, and later by the helical electrode 24 in combination with tissue encapsulation that naturally develops around the implanted device.

FIG. 10C shows a side plan of the lead body 22 in more detail. In FIG. 10C, the lead body 22 includes an entry slot 76 near the proximal end 18 and an exit slot 78 near the distal end 20. The tether 66 is inserted through the entry slot 76, passes through the lumen 70, and exits through the exit slot 78. The exit slot 78, in one embodiment, allows the electrode 24 to be inserted into the epicardium 26 at the location 74 (shown in FIG. 10B) without drawing the tether 66 into the epicardium 26. In this embodiment, the exit slot 78 may be located from about 1 to about 10 mm from the distal end 20. In another embodiment, the exit slot 78 is located at the distal end 20.

FIGS. 10D-10E show a side plan and an end view of another embodiment of the lead body 22. The lead 22 includes the lumen 70, the entry slot 76, the exit slot 78, and a region 80 extending from the exit slot 78 to the distal end 20 of the lead body 22. The region 80 includes a longitudinal score line 80 adjacent to the lumen 70. As the lead 22 is advanced to the anchor 64, the tether 66 extends through the lumen 70 to the distal end 20 of the lead body 22. This embodiment allows the lead body 22 to be more easily advanced to the anchor 64 while avoiding drawing the tether 66 into the epicardium 26 as the electrode 24 is inserted. In the embodiments shown in FIGS. 10A-10E, the lead body 22 is advanced over the tether 66, but alternatively, the tether 66 could be eliminated and the lead body 22 advanced over an adhesive catheter. FIG. 10F shows the lead body 22 after it has been advanced to the location 74 and the electrode 24 has been inserted into the epicardium 26.

In an alternative embodiment, the electrode 24 comprises a plunge electrode with tines. In this embodiment, the electrode 24 penetrates the epicardium 26. Once the epicardium 26 is penetrated, the electrode 24 is pulled back and the tines act on the inside of the epicardium 26 to retain the electrode 24 in a desired position.

FIG. 11 shows an exemplary method 100 of using the lead 14 of FIGS. 10A-10F. The attachment system 62 including the anchor 64 and the tether 66 are inserted into the patient (block 110). Next, adhesive 44 is delivered to the anchor 64 (block 120). This can be accomplished using an adhesive catheter, blister packs, or some other method. The anchor 64 and attached tether 66 are placed at the desired location on the epicardium 26 (block 130). A guide catheter can be used to insert and place the anchor 64 and tether 66. After the anchor 64 is bonded to the epicardium 26 by adhesive 44, the lead body 22 is advanced toward the heart 16 over the tether 66 in the direction y to a location 74 distal to the anchor 64 (block 140). The lead 14 is then attached to the epicardium 26 (block 150) by using, for example, a helical electrode 24 to puncture and couple to the epicardium 26. If the embodiment shown in FIGS. 10D-10E is used, once the lead is advanced to a desired position but prior to the attachment of the electrode, the tether 66 is pulled through score line 81 so that it exits the lead body 22 at the exit slot 78.

In an alternative embodiment, the method can further include verifying the electrical characteristics of the lead 14, the heart 16, and electrode 24 before permanently attaching the electrode 24. The anchor 64 and tether 66 allow the electrode 24 to be placed in a generally desired region on the heart 16. The clinician can, for example, use the position of the tether 66 relative to a landmark on the proximal end of the lead (e.g., a terminal) to help control the point of penetration into the epicardium 26. In this embodiment, the tether 66 may include markings on a proximal end to assist the clinician in determining this relative position. These marking can be used to constrain the penetration site to a circular region about the anchor 64.

In one embodiment, the clinician then verifies the electrical characteristics of the heart 16, the lead 14, and the electrode 24. In this embodiment, the clinician can use the markings on the tether 66 to help gauge the degree of penetration of the electrode 24 into the heart 16. The clinician may, for example, select a first site and advance the electrode 24 a small distance (e.g., about 1 mm) through the epicardium 26. The clinician then tests whether the pacing thresholds are suitable. If the electrode 24 is not in a suitable location, it can be withdrawn and repositioned. Conversely, if the electrode 24 location is suitable, it can be advanced further into the heart 16 to a final attachment position (e.g., between about 1 and 10 mm), again using the markings to gauge the penetration distance.

Various modifications and additions can be made to the exemplary embodiments discussed without departing from the scope of the present invention. Accordingly, the scope of the present invention is intended to embrace all such alternatives, modifications, and variations as fall within the scope of the claims, together with all equivalents thereof.

We claim:

1. A lead for electrically communicating with a human heart, the lead comprising:
a flexible, elongated lead body having a proximal end and a distal end;
a pad having an interface surface coupled to the lead body at the distal end;
an electrode coupled to the pad; and
a delivery means for delivering an adhesive to the interface surface after the lead has been inserted into a subject,
wherein the adhesive bonds the pad to the heart, such that the electrode is electrically coupled to the heart.

2. The lead of claim 1 wherein the delivery means is a lumen extending from the proximal end of the lead body to the distal end of the lead body, the lumen adapted to transport the adhesive to the interface surface.

3. The lead of claim 2 further including a catheter adapted for insertion into the lumen, the catheter adapted to transport the adhesive to the interface surface.

4. The lead of claim 2 wherein the delivery means includes at least one encapsulation pocket disposed on the pad and adapted to rupture and release the adhesive to the interface surface in response to the application of pressure.

5. The lead of claim 4 wherein the encapsulation pocket comprises a material selected from the group consisting of: a gelatin, a collagen, a starch, or a polysaccharide.

6. The lead of claim 1 wherein the pad includes a recess adapted to retain the adhesive.

7. The lead of claim 1 wherein the electrode comprises a porous material adapted for absorbing the adhesive.

8. The lead of claim 7 wherein the adhesive comprises an electrically conductive adhesive.

9. The lead of claim 1 wherein the delivery means includes a catheter adapted to deliver the adhesive to the interface surface after the lead has been inserted into the subject.

10. The lead of claim 1 further comprising a pulse generator in communication with the electrode, the pulse generator adapted to generate an electrical therapy.

11. A method of electrically coupling a cardiac rhythm management system, including a pulse generator and a cardiac lead, to a human heart, the method comprising:
providing a cardiac lead having a flexible, elongated lead body having a proximal end, a distal end, and an electrode coupled to the lead body near the distal end, the lead having an interface surface near the distal end adapted for interfacing with the heart;
inserting the lead into the subject;
delivering an adhesive to the interface surface near the electrode, after inserting the lead; and
bonding the interface surface to the heart, such that the electrode is electrically coupled to an epicardial surface of the heart.

12. The method of claim 11 wherein the delivering step includes delivering the adhesive through a lumen extending from the proximal end to the distal end.

13. The method of claim 12 wherein the delivering step includes inserting a delivery catheter through the lumen and delivering the adhesive through the delivery catheter.

14. The method of claim 11 wherein the electrode comprises a porous material adapted for absorbing the adhesive and further wherein the adhesive is delivered to the electrode.

15. The method of claim 11 wherein the pad includes an encapsulation pocket adapted to rupture and release the adhesive to the interface surface in response to the application of pressure, and wherein the delivery step includes applying pressure to the pad sufficient to rupture the encapsulation pocket and thereby release the adhesive to the interface surface.

16. A lead for electrically communicating with a human heart, the lead comprising:
a flexible, elongated lead body having a proximal end and a distal end, the lead body having an exit slot located near the distal end and a lumen extending from the proximal end to the exit slot;
an electrode coupled to the distal end of the lead body;
a tether having a tether proximal end and a tether distal end, the tether extending through the lumen; and
an anchor having an interface surface adapted for coupling to the heart using an adhesive, the anchor coupled to the tether distal end.

17. The lead of claim 16 wherein the lumen extends to the distal end of the lead body and the lead body further comprises a score line adjacent to the lumen and extending from the distal end to the exit slot.

18. The lead of claim 16 wherein the electrode comprises a helical electrode.

19. The lead of claim 18 wherein the electrode further comprises an extendable/retractable helical electrode.

20. The lead of claim 16 wherein the tether includes a plurality of reference markings.

21. The lead of claim 16 wherein the exit slot is disposed at a location of from about 1 to about 10 mm from the distal end of the lead body.

22. The lead of claim 16 wherein the anchor includes an encapsulation pocket adapted to rupture and release the adhesive to the interface surface in response to the application of pressure.

23. A method of electrically coupling a cardiac pacing lead to a human heart comprising:
providing a cardiac lead having a flexible, elongated lead body having a proximal end and a distal end, a lumen extending longitudinally through the lead body, and an electrode coupled near the distal end of the lead body;
positioning an anchor on an epicardium of the heart, the anchor coupled to a longitudinally extending tether and having an interface surface adapted for coupling to the heart using an adhesive;
delivering the adhesive to the interface surface to bond the anchor to the epicardium;
advancing the cardiac lead over the tether toward a location on the heart near the anchor; and
attaching the electrode to the heart.

24. The method of claim 23 wherein the tether includes a plurality of reference markings, and further wherein the advancing step includes determining the location by using the reference markings.

25. The method of claim 24 wherein the method further comprises verifying an electrical characteristic after the lead body is advanced to the location near the anchor and repositioning the electrode if necessary prior to attaching the electrode.

26. The method of claim 25 wherein the attaching step includes determining a penetration depth of the electrode using the reference markings.
